(19) United States
(12) Patent Application Publication
(10) Pub. No.: US 2011/0212460 A1
(43) Pub. Date: Sep. 1, 2011

(54) ASSAY FOR NITROUS OXIDE NEUROLOGIC SYNDROME

(76) Inventors: Kirk J. Hogan, Madison, WI (US); Rebecca M.R. Selzer, Verona, WI (US)

(21) Appl. No.: 13/012,561

(22) Filed: Jan. 24, 2011

Related U.S. Application Data
(63) Continuation of application No. 11/234,425, filed on Sep. 23, 2005, now abandoned.
Application No. 11/234,425 is a division of application No. 10/373,131, filed on Feb. 24, 2003, now abandoned.
(60) Provisional application No. 60/358,781, filed on Feb. 22, 2002.

(51) Int. Cl. C12Q 1/68; G01N 33/573; C07H 21/04

(52) U.S. Cl. 435/6.18; 435/7.4; 536/24.31

(57) ABSTRACT

A method for detection of susceptibility to nitrous oxide neurologic syndrome in a subject is disclosed. In one embodiment, the method comprises: (a) providing a sample from a subject, wherein said subject is a candidate for nitrous oxide anesthesia; and (b) detecting the presence or absence of folate, cobalamin, methionine and homocysteine pathway genetic polymorphisms in said sample, wherein the presence of a polymorphism indicates that the subject is susceptible to nitrous oxide neurologic syndrome.

### CROSS-REFERENCE TO RELATED APPLICATION

The present invention claims priority to U.S. Ser. No. 60/358,781, incorporated by reference herein.

### STATEMENT REGARDING FEDERALLY SPONSORED RESEARCH OR DEVELOPMENT

### Background of the Invention

Nitrous oxide irreversibly oxidizes the cobalt atom of vitamin B₁₂, thereby inhibiting activity of the cobalamin-dependent enzyme methionine synthase (5-methyltetrahydrofolate-homocysteine methyltransferase, MTR, EC.2.1.1.13). Methionine synthase catalyses the re-methylation of 5-methyltetrahydrofolate and homocysteine to tetrahydrofolate and methionine which, via its activated form S-adenosylmethionine, is the principal substrate for methylation in many biochemical reactions including assembly of the myelin sheath, neurotransmitter substitutions, and DNA synthesis in rapidly proliferating tissues (FIG. 1) (Chiang, P. K., et al., Faseb J. 10:471-80, 1996).

5,10-methylene tetrahydrofolate reductase (MTHFR) regulates the synthesis of 5-methyl tetrahydrofolate, the primary circulatory form of folate which acts as the methyl donor to methionine. Homocysteine is a sulphur amino acid formed by demethylation of the essential amino acid methionine. A methyltransferase enzyme known as methionine synthase (MTR) is responsible for converting homocysteine back to methionine, the body's sole methyl donor. Among many other reactions, methyl moieties are crucial for the synthesis of neurotransmitters, assembly of the myelin sheath, and DNA synthesis in proliferating tissues such as bone marrow and the developing brain. Genetic defects that cause deficiencies in either MTR or MTHFR are associated with high serum homocysteine levels and homocystinurea. Nitrous oxide irreversibly oxidizes the cobalt atom of vitamin B₁₂, and thus inhibits the activity of the cobalamin-dependent enzyme MTR.

Over twenty-four rare mutations in MTHFR have been described as associated with pronounced enzymatic deficiency and homocystinuria. In addition, two common single nucleotide polymorphisms have been identified that affect folate and homocysteine metabolism, both of which are implicated in the pathogenesis of cardiovascular disease, neural tube defects and developmental delay. One polymorphism is a missense mutation consisting of a C→T transition at position 677, which produces an alanine to valine amino acid substitution within the catalytic domain of MTHFR. The resulting enzyme has reduced catalytic activity. The second mutation is found at position 1298, an A→C transition which results in a glutamate to alanine substitution located in the presumed regulatory domain of MTHFR.

Methionine synthase inactivation by nitrous oxide has been demonstrated with purified enzyme (Frasca, V., et al., J. Biol. Chem. 261:15823-6, 1986), in cultured cells (Christensen, B., et al., Pediatr. Res. 35:3-9, 1994; Fiskerstrand, T., et al., J. Pharmacol. Exp. Ther. 282:1305-11, 1997), experimental animals (Kondo, H., et al., J. Clin. Invest. 67:1270-83, 1981), and humans (Koblin, D. D., et al., Anesth. Analq. 61:75-8, 1982; Royston, B. D., et al., Anesthesiology 68:213-6, 1988; Christensen, B., et al., Anesthesiology 80:1046-56, 1994). The mean half-time of inactivation is 46 minutes. Residual methionine synthase activity following greater than 200 minutes of nitrous oxide administration approaches zero (Royston, B. D., et al., supra, 1988). Mice, pigs, and rats exposed to nitrous oxide demonstrate delayed recovery of enzyme activity over 4 days or longer (Kondo, H., et al., supra, 1981; Deacon, R., et al., Eur. J. Biochem. 104:419-23, 1980; Molloy, A. M., et al., Biochem. Pharmacol. 44:1349-55, 1992; Koblin, D. D., et al., Anesthesiology 54:318-24, 1981). Recovery in cultured cells indicates that nitrous oxide-mediated inhibition is irreversible, with de novo synthesis of the enzyme required to restore activity (Riedel, B., et al., Biochem. J. 341:133-8, 1999).

Severe MTHFR deficiency is an autosomal recessive disorder characterized by progressive hypotonia, convulsions and psychomotor retardation. The clinical presentation may be subtle, manifesting as developmental disability in the setting of moderate homocystinuria and hyperhomocystinemia, and low to normal levels of plasma methionine (Rosenblatt, D. S. and Fenton, W. A., supra, 2001). At least twenty-nine mutations in MTHFR are associated with severe deficiency (usually 0-30% of control activity) (Goyette, P., et al., supra, 1994; Goyette, P., et al., Am. J. Hum. Genet. 59:1268-75, 1996; Goyette, P., et al., Am. J. Hum. Genet. 56:1052-9, 1995; Kluijtmans, L. A., et al., Eur. J. Hum. Genet. 6:257-65, 1998; Sibani, S., et al., Hum. Mutat. 15:280-7, 2000; Tonetti, C., et al., J. Inherit. Metab. Dis. 24:833-42, 2001; Homberger, A., et al., J. Inherit. Metab. Dis. 24:50(Suppl. 1), 2001). The preponderance of patients are compound heterozygotes for distinct MTHFR substitutions, with a small minority representing allelic homozygotes.

### SUMMARY OF THE INVENTION

In one embodiment, the present invention is a method for detection of susceptibility to nitrous oxide neurologic syndrome in a subject, comprising providing a sample from a subject, wherein said subject is a candidate for nitrous oxide exposure; and detecting the presence or absence of folate, cobalamin, methionine and homocysteine pathway genetic polymorphisms in said sample, wherein the presence of a polymorphism indicates that the subject is susceptible to nitrous oxide neurologic syndrome. Preferably, the sample is selected from the group consisting of a blood sample, a tissue sample, a urine sample, a cerebrospinal fluid sample, and an amniotic fluid sample and the subject is selected from the group consisting of an embryo, a fetus, a newborn animal, a young animal, and a mature animal. Most preferably, the subject is human.

In one embodiment, the detecting of step (b) is genomic testing. In a specific embodiment, genomic testing is testing for MTHFR polymorphisms preferably 1755G→A. In another embodiment, the said genomic testing is testing for polymorphisms in the methionine synthase, methionine synthase reductase, and cystathionine β-synthase genes.

In another embodiment, the detecting is based on observations of peptides or proteins in the pathway, preferably via an enzyme activity assay or via the assay of a metabolite of the pathway.

The present invention is also a kit comprising a reagent for detecting the presence or absence of folate, cobalamin, methionine and homocysteine pathway genetic polymorphisms in a sample, wherein the reagent is a nucleic acid molecule comprising at least 11 nucleotides of the MTHFR, MTR, MTRR or CBS genes or their complement and preferably, further comprising instructions for using said kit for detecting the presence or absence of folate, cobalamin, methionine and homocysteine pathway genetic polymorphisms in a sample.

In another embodiment, the invention is a method of diagnosing 5,10-methylene tetrahydrofolate reductase deficiency in a human patient comprising examining a patient's 5,10-methylene tetrahydrofolate reductase gene and determining whether a polymorphism exists in residue 1755, preferably 1775 G→A.

Other embodiments of the invention will be apparent to one of skill in the art after examination of the specification claims and drawings.

### BRIEF DESCRIPTION OF THE SEVERAL VIEWS OF THE DRAWING

FIG. 1 illustrates the folate/homocysteine metabolic cycles and enzymatic site of nitrous oxide toxicity. MTR, methionine synthase; MTRR, methionine synthase reductase; CBS, cystathionine β-synthase; MTHFR, 5,10-methylenetetrahydrofolate reductase.

FIG. 2 illustrates nucleotide changes in the MTHFR gene of the patient and his parents. In addition to the coding changes, the proband and his mother are heterozygous for a C→A substitution at position 2355, 375 bases 3′ of the stop codon, on the same chromosome as the 1298C polymorphism.

FIG. 3 discloses MTHFR exon 10 mRNA sequence (SEQ ID NO:1) flanking a G1755A polymorphism, along with exon 11 mRNA sequence (SEQ ID NO:2), which would be expressed 3′ of the exon 10 MTHFR mRNA and intronic sequence immediately 3′ to Exon 10 (SEQ ID NO:3). The site of the G1775A polymorphism is underlined.

### DETAILED DESCRIPTION OF THE INVENTION

Applicants have investigated an infant's neurologic deterioration and death after anesthesia with nitrous oxide. Applicants have discovered a novel mutation at base pair 1755 and exon 10 of the human MTHFR gene which caused the neurological syndrome. This G→A transition results in a substitution of an isoleucine residue for a methionine residue at the amino acid 581 of the MTHFR protein. This mutation represents a newly discovered pharmacogenetic syndrome, identified as neurological deterioration after nitrous oxide exposure in genetically predisposed subjects.

In one embodiment, the present invention is a method for detection of susceptibility to nitrous oxide neurologic syndrome. As used herein, the term “nitrous oxide neurologic syndrome” refers to neurologic deterioration after nitrous oxide exposure in a genetically susceptible subject manifested clinically by, but not limited to, lethargy, paresthesia, hypotonia, hyporeflexia, reduced level of consciousness, and incoordination. Signs and symptoms of nitrous oxide neurologic syndrome may be mild, moderate or severe in presentation. Findings of nitrous oxide neurologic syndrome on cranial computed tomography and magnetic resonance imaging may include, but are not limited to, generalized brain and spinal cord atrophy. Findings of nitrous oxide neurologic syndrome on post-mortem examination may include, but are not limited to, nervous system atrophy and demyelination.

In one embodiment, the method comprises providing a sample from the subject wherein the subject is a candidate for nitrous oxide anesthesia and detecting the presence or absence of folate, cobalamin, methionine and homocysteine pathway genetic polymorphisms in the sample. By “folate, cobalamin, methionine and homocysteine pathway,” we mean genes and gene products involved in the synthesis of these metabolites. Mudd, et al. (Mudd, S. H., et al., “Disorders of Transsulfuration,” In: Scriver, C. R., et al., eds. The Metabolic and Molecular Bases of Inherited Disease, Vol. 1: McGraw-Hill, 2007-2053, 2001) and Rosenblatt, D. S. and Fenton, W. A. (“Inherited Disorders of Folate and Cobalamin Transport and Metabolism,” In: Scriver, C. R., et al., eds., The Metabolic and Molecular Bases of Inherited Disease, Vol. 1: McGraw-Hill, 3897-3933, 2001), both incorporated by reference, disclose the pathways and constituents. The presence of a polymorphism indicates that the patient is susceptible to nitrous oxide neurologic syndrome, and that safer alternative anesthetic agents and regimens may be considered. Nitrous oxide exposure could still be suitable if benefits to the exposure are outweighed by risks of non-exposure.

As used herein, the term “candidate of nitrous oxide exposure” refers to a subject for whom knowledge of susceptibility to nitrous oxide neurologic syndrome would be a factor in deciding whether or not to administer nitrous oxide.

In a preferred version, the sample is selected from the group consisting of a blood sample, a tissue sample, a urine sample, a cerebrospinal fluid sample, and an amniotic fluid sample. The subject may be an animal, preferably a human animal, of any age but is preferably newborn or young animal. If the subject is a human, the subject is preferably less than 12 years old. In another embodiment of the invention, the subject is an embryo or a fetus.

In another version, the patient has already been exposed at least once to nitrous oxide.

### Candidate Genes for Genetic Polymorphisms Causing Nitrous Oxide Neurologic Syndrome

In one preferred embodiment of the present invention, one would analyze the patient sample by genomic testing. A preferred genomic testing protocol would be to examine various genes in the folate, cobalamin, methionine and homocysteine pathway for polymorphisms. The following are representative and preferred enzymes/gene products of the genes.

TABLE 1

| | Reference | |
|---|---|---|
| | (GenBank | MIM |
| Gene | Number) | Numbers |
| 5, 10 Methylene tetrahydrofolate reductase | NM_005957 | MIM 607093 |
| Methionine synthase | NM 000254 | MIM 156570 |
| Methionine synthase reductase | NM 002454 | MIM 602568 |
| Glutamate formiminotransferase | | MIM 229100 |
| Dihydrofolate reductase | | MiM 126060 |
| Methenyl tetrahydrofolate cyclohydrolase | | MIM 604887 |
| Methyltetrahydrofolate homocysteine | | |
| methyltransferase | | |
| Mitochondrial Cbl reductase | | |
| Cob(I)alamin adenosyltransferase | | |
| Cytosolic Cbl reductase/β-ligand transferase | | |
| Cystathionine β-synthase | NM 000071 | MIM 236200 |
| Methionine adenosyltransferase | | MIM 250850 |
| γ-Cystathionase | | MIM 219500 |

The “GenBank number” would lead one to the GenBank sequence of the particular gene. The “MIM number” is a citation to the “Mendelian Inheritance in Man” accession number, which leads one to references describing known polymorphisms, and links cited therein to exonic and genomic sequences and to the GenBank sequence.

One would examine a candidate patient sample for polymorphisms in any of the listed genes, most preferably in 5,10-methylene tetrahydrofolate reductase, methionine synthase reductase, methionine synthase, and cystathionine β-synthase.

To determine whether the listed genes comprise a polymorphism, one would compare the patient's gene sequence with that of the standard or reference sequence referenced above by means known to one of skill in the art. Various means are described below and in the Examples.

### Phenotypic Tests for Genetic Polymorphisms Causing Nitrous Oxide Neurologic Syndrome

One may also wish to examine the phenotype of a test subject for genetic polymorphisms. Phenotypic indicators of genetic polymorphisms causing nitrous oxide neurologic syndrome include, but are not limited to, enzyme assays and increase or decrease of a pathway metabolite. Decrease of enzyme activity would indicate a susceptibility to the syndrome.

For example, MTHFR activity in cultured fibroblasts below the normal range (normal 13.3±4.6 nmoles HCHO/mg protein/h) would be diagnostic of genetic susceptibility to the syndrome. Similarly, one would examine the sample for elevated total serum homocysteine (normal 5.4-13.9 υM), presence of homocystine in the urine (normal 0.0), and/or depressed plasma methionine (normal 0.48±0.18 mg/dl).

### MTHFR Gene Mutations

In one preferred embodiment, the present invention is a method of screening for a particular mutation in the MTHFR gene. The Examples disclose Applicants' recent discovery of the novel mutation and should be examined in their entirety for further explanation and disclosure relevant to the present invention. In one embodiment, one would attempt to diagnose children with general metabolic signs of the disorder (e.g., hypotonia, muscular tone abnormalities, seizures). In another embodiment, one would attempt to diagnose individuals who are about to be exposed to nitrous oxide anesthesia.

The diagnosis would involve examining the MTHFR gene of the patient and determining whether a mutation at position 1755 has occurred, preferably 1755 G→A. This examination may take place as described in the Examples or by other appropriate equivalent genotyping methods known to those of skill in the art.

One may find the sequence of the MTHFR gene at Genbank accession number NM_{—}005957. In a preferred method of the present invention, one would amplify a DNA sample from a patient or reverse transcribe an RNA sample from the patient into DNA and amplify the DNA. One would then analyze the amplified DNA to determine whether the sample comprises a mutation in residue 1755 of the gene.

In the numbering system used herein, “residue 1755” corresponds to the standard numbering system for the gene. A reference to the standard MTHFR numbering system, and the one which we have adopted, is Goyette, P., et al., Mammalian Genome 9:652-656, 1998, incorporated by reference.

In a preferred method of the present invention, one would also examine the MTHFR gene for other sequence abnormalities known to be indicative of MTHFR deficiency. U.S. Pat. Nos. 6,218,170 and 6,074,821, incorporated by reference, list such abnormalities. One would particularly wish to examine the sequence for the presence or absence of the 677C→T and 1298A→C mutations. Other polymorphisms are available at MIM 607093.

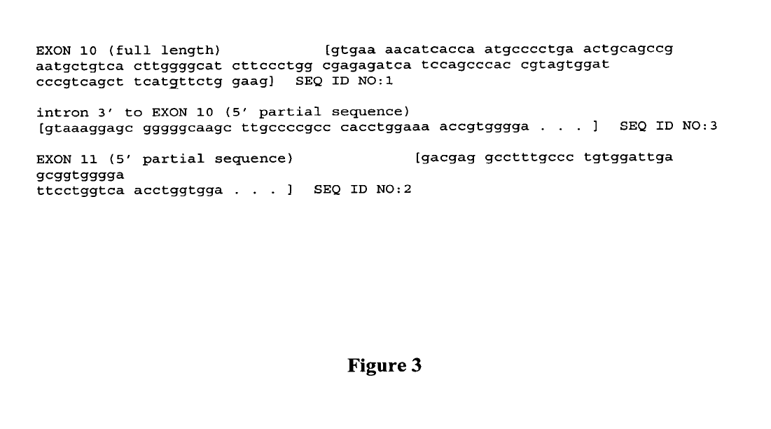

The present invention is also a probe designed to detect the mutation in residue 1755. Preferably, this probe comprises a nucleic acid identical or complementary to a fragment of the MTHFR gene comprising residue 1755. In one embodiment, the probe would comprise a sequence identical or complementary to the mutated residue. One of ordinary skill could examine FIG. 3, a figure comprising the MTHFR mRNA sequence and flanking genomic sequences and expressed sequences, to construct such a probe. Preferably, such a probe would comprise the sequence or complementary sequence within 5 nucleotides of each side of the 1755 polymorphism.

If one wished to use a genomic probe, the sequences or complementary sequences selected from the “Exon 10” sequence (SEQ ID NO:1) of FIG. 3 may be combined with the intron sequence listed in FIG. 3. For a cDNA/mRNA probe, Exon 10 sequences may be continuous with the Exon 11 sequences listed in FIG. 3. The description of preferred probes in the section below lists the sizing of preferred probes.

### Kits

The present invention also comprises kits comprising reagents for detecting the presence or absence of genetic polymorphisms in the pathways described above. In one preferred embodiment, the reagent would be a nucleic acid. In different embodiments, the nucleic acid would be selected from the group of less than 10 nucleotides in length, between 10-15 nucleotides in length and greater than 15 nucleotides in length. In one embodiment, the nucleic acid is identical or complementary to the wild-type sequence and in another embodiment the nucleic acid is identical or complementary to the mutant sequence.

The table below describes preferred sequences. The sequences listed may be used as noted or as the complement of the noted sequence. A suitable probe will comprise the listed sequences but may have additional sequences on either end. Particularly preferred additional sequences are listed in FIG. 3 and Table 2.

TABLE 2

| | Intended Target |
|---|---|
| Probe | Sequence |
| ttcatgttctg | MTHFR gene |
| cccgtcagcttcatgttctggaag | MTHFR gene |
| ttcatattctg | MTHFR gene |
| ccgtcagcttcatattctggaac | MTHFR gene |

In another embodiment, the reagent is selected from the group consisting of enzymes, enzyme inhibitors or enzyme activators. The kit may comprise chromatographic compounds, fluorometric compounds and/or spectroscopic labels. The kit may also contain a radioisotope.

Preferred enzyme, enzyme inhibitors or enzyme activators would include restriction endonucleases (e.g. NlalII, HinfI, MbolI), FEN-1 cleavases, and ligases.

### Examples

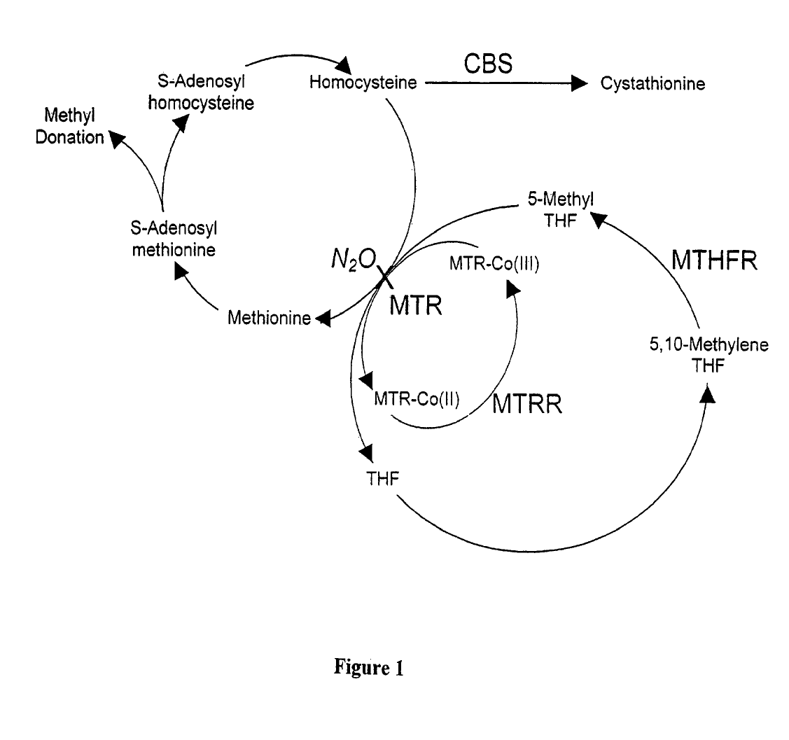

A child discovered to have 5,10-methylenetetrahydrofolate reductase (MTHFR, EC.1.1.1.68) deficiency (OMIM #236250) died after two anesthetics using nitrous oxide (Beckman, D. R., et al., Birth Defects Orig. Artic. Ser. 23:47-64, 1987). MTHFR catalyzes the synthesis of 5-methyltetrahydrofolate. Sequence analysis of RNA transcripts and genomic DNA from the patient and family members, together with direct assays of fibroblast MTHFR activity, reveal that the enzyme deficiency was caused by a novel MTHFR mutation (1755G→A) which changes conserved methionine 581 to an isoleucine, co-inherited with two common MTHFR polymorphisms (677C→T, 1298A→C) each associated with depressed enzyme function. (Frosst, P., et al., Nat. Genet. 10:111-3, 1995; van der Put, N. M., et al., Am. J. Hum. Genet. 62:1044-51, 1998). A nitrous oxide-induced defect of methionine synthase superimposed on an inherited defect of MTHFR (FIG. 1) caused the patient's death.

### Case Report

The patient was normal until 3 months of age when a mass was noted on the left lower extremity. Although not recognized prior to the patient's surgery, both the father and uncle have serum total homocysteine levels >30.0 μM (normal 5.4-13.9 μM). On life-long, high dose vitamin B supplements, the proband's sibling has a homocysteine level of 4.3 μM. Neither the father nor the sibling has received nitrous oxide. On preoperative assessment for excisional biopsy of the tumor the patient was American Society of Anesthesiologists status I. After atropine premedication, and sodium thiopental and succinylcholine induction, the trachea was intubated and anesthesia maintained with 0.75% halothane and 60% nitrous oxide in oxygen for 45 minutes.

Surgical resection of the mass was scheduled for the fourth day after the biopsy. Following a halothane inhalational induction, the child was anesthetized for 270 minutes with 0.75% halothane and 60% nitrous oxide. At the conclusion he was extubated and transferred awake to the ICU. He was discharged on the seventh postoperative day in apparent good health. Seventeen days later he was admitted for seizures and episodes of apnea. Examination revealed a severely hypotonic infant with absent reflexes and ataxic ventilation. Cranial computed tomography showed generalized atrophy of the brain with enlarged prepontine and medullary cisterns. The urine was positive for homocystine (1.30 umol/mg creatinine, normal 0), but negative for organic acids and methylmalonic acid. In the plasma, a homocystine level of 0.6 mg/dl (normal <0.01) and methionine level of 0.06 mg/dl (normal 0.48±0.18) were found, with a vitamin B₁₂ level of 403 pg/ml (normal range 150-800 pg/ml). The serum folate level by RIA was 3.8 ng/ml (normal 2.5-15 ng/ml), with a CSF folate of 26 ng/ml (normal 10.6 to 85 ng/ml.).

The patient died at 130 days of age after respiratory arrest. The autopsy showed asymmetric cerebral atrophy and severe demyelination, with astrogliosis and oligodendroglia) cell depletion in the mid-brain, medulla and cerebellum. MTHFR activity in cultured fibroblasts reported post-mortem was 1.22 nmol formaldehyde (HCHO) produced/h/mg protein (normal 5.04±1.36) with flavinadenine dinucleotide (FAD), and 0.8 without. Simultaneous control values were 6.4 and 5.4 with and without FAD, respectively (P. Wong, Chicago, Ill.). (Kanwar, Y. S., et al., Pediatr. Res. 10:598-609, 1976.)

### Methods:

### Fibroblast Culture and MTHFR Activity

Fibroblasts were cultured from the parents' skin punch biopsies and from the proband's stored samples. MTHFR activity was measured at confluence as previously described. (Rosenblatt, D. S. and Erbe, R. W., Pediatr. Res. 11:1137-41, 1977). All assays were performed in duplicate with a simultaneous normal control.

### Genomic DNA Preparation and Sequence Analysis

Genomic DNA was isolated from cultured fibroblasts from the patient and both parents, and from either blood or buccal cells from other relatives. Each of the 11 MTHFR exons was amplified from genomic DNA by PCR using newly designed intronic primers (see Table 4). PCR products were bi-directionally sequenced in the parents and proband. A novel mutation in the proband at nucleotide 1755 (exon 10), and two previously described frequent polymorphisms at positions 677 (exon 4) and 1298 (exon 7) in the MTHFR gene, were analyzed in genomic DNA from the parents and other relatives using NlalII, HinfI, and MbolI as previously described (Frosst, P., et al., supra, 1995; van der Put, N. M., et al., supra, 1998). Family members were also screened as previously described for common polymorphisms in the genes encoding enzymes regulating folate and homocysteine metabolism implicated in the pathogenesis of neural tube defects, other congenital anomalies, and cardiovascular and neoplastic disease (Schwahn, B. and Rozen, R., Am. J. Pharma. 1:189-201, 2001), including MTR (D919G) (Harmon, D. L., et al., Genet. Epidemiol. 17:298-309, 1999), MTRR (I22M) (Wilson, A., et al., Mol. Genet. Metab. 67:317-23, 1999), and CBS (68-bp duplication) (Tsai, M. Y., et al., Am. J. Hum. Genet. 59:1262-7, 1996).

### RNA Analysis

To evaluate expression of an intact copy of the predominant 7.2 kb MTHFR isoform (Gaughan, D. J. et al., Gene 257:279-89, 2000), RNA was isolated from the proband's cultured fibroblasts. A 2206 bp product containing the entire coding region was amplified by PCR from the cDNA transcript and sequenced in full (primers in Table 2). The 7.2 kb cDNA product was amplified as seven overlapping fragments (primers in Table 2) ranging from 1.0-2.2 kb as verified by gel electrophoresis. Bands corresponding to expected fragment sizes were excised, and the first 300 bases of the 5′- and 3′-ends were sequenced to positively identify each fragment. Fragments from the proband and an unrelated control were then compared.

### Results:

### Enzyme Activity in Fibroblasts

The patient's MTHFR activity in two replicates was 0.76 and 0.03 nmoles HCHO/mg protein/h (normal range of 13.3±4.6 nmoles HCHO/mg protein/h), with a simultaneous normal control of 11.52 nmoles HCHO/mg protein/h. MTHFR activities in the father (1.8 nmoles HCHO/mg protein/h) and mother (6.1 nmoles HCHO/mg protein/h) were reduced, with a control level of 9.5 nmoles HCHO/mg protein/h.

### Genomic DNA-Sequence Analysis

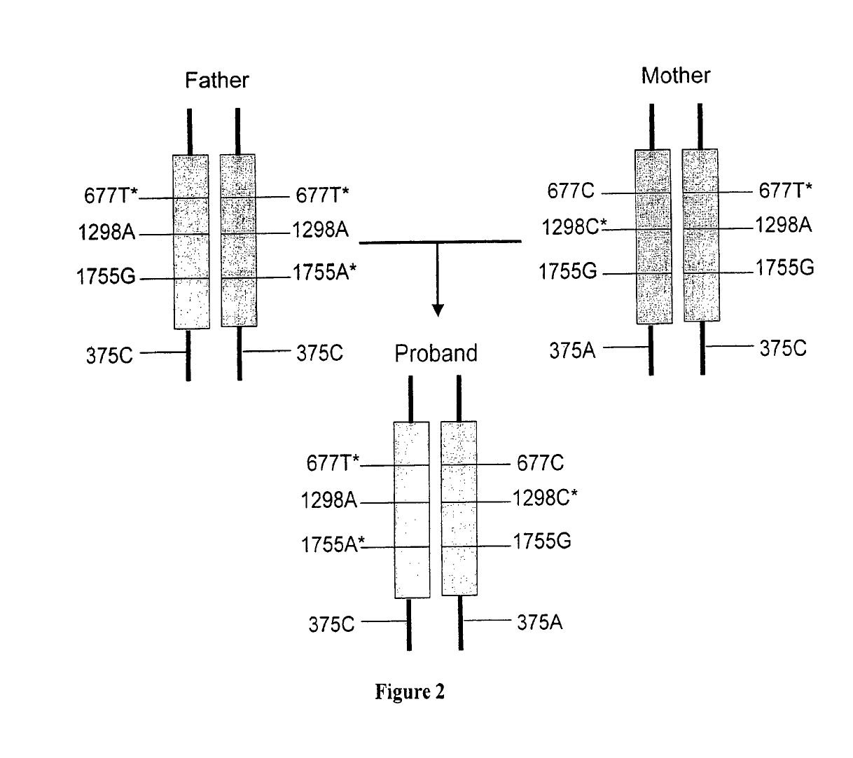

The patient was found to be heterozygous for a novel mutation, 1755G→A in exon 10, which produces a methionine to isoleucine substitution (M581I) (Goyette, P., et al., Nat. Genet. 7:551, 1994) (Genbank accession number NM_{—}005957). Restriction enzyme analysis confirmed presence of the 1755G→A mutation in the heterozygous patient, his father, his brother, one uncle and one aunt, but not in 100 control chromosomes. The patient was also heterozygous for 677C→T in exon 4 (A222V) and 1298A→C in exon 7 (E429A). In addition to being heterozygous for 1755G→A, the father is homozygous TT for 677C→T and homozygous AA for 1298A→C (FIG. 2). The mother is heterozygous for both common polymorphisms, and homozygous wild type at 1755G→A. The patient's sib has an identical haplotype to the patient in all coding regions. The novel mutation at 1755G→A has therefore been transmitted to the patient from a paternal chromosome in cis with the 677C→T mutation. Two of the father's four siblings have identical haplotypes to the father, exhibiting the heterozygous 1755G→A and homozygous 677C→T mutations (Table 1).

25-40 bases beyond all intronic boundaries were sequenced to detect possible altered splice junctions. 5′- and 3′-UTR regions flanking the MTHFR gene revealed no substitutions within or proximate to a putative binding site for a transcription factor or an actual start site as mapped by Gaughan, et al. (supra, 2000) and Homberger, et al. (Homberger, A., et al., Eur. J. Hum. Genet. 8:725-9, 2000). Sequence approximately 550 bp 3′ from the MTHFR stop codon and 400 bp encompassing the distal 3′-polyadenylation site exhibited several polymorphisms but none at sites with recognized functional significance.

### MTR, MTRR, CBS Genomic Analysis

Genotypes at these loci for all members of the pedigree are provided in Table 3.

### RNA Analysis

No size differences of the 7 MTHFR cDNA fragments were observed, indicating that the patient's fibroblasts express an intact MTHFR transcript. The 2.2 kb product contained the entire coding region of the transcript and was used to sequence 50 bp 5′ to the translational start site to 150 bp downstream of the stop codon. This product was of the expected length, and no alternate splicing variants were detected. The entire product was sequenced and compared to the published sequence (Harmon, D. L., et al., supra, 1999) (Genbank accession number NM_{—}005957). The heterozygous common polymorphisms 677C→T and 1298A→C, as well as the heterozygote substitution 1755G→A, were confirmed.

The proband's 1755G→A substitution occurs in a phylogenetically conserved region of the MTHFR protein [BLASTP 2.2.1]. This region, which is thought to be essential for functional protein folding (Goyette, P. and Rozen, R., Hum. Mutat. 16:132-8, 2000), is a mutational “hotspot” for MTHFR deficiency (1711C→T, 1727C→T, 1762A→T, 1768G→A) (Kluijtmans, L. A., et al., supra, 1998; Sibani, S., et al., supra, 2000). Heterozygous presence of the substitution in the proband's father, brother, uncle and aunt, but its absence in 100 independent control chromosomes, suggests that it is not a benign variant.

Compound heterozygosity for common MTHFR alleles 677C→T and 1298A→C, as seen in the patient, mother, and brother, causes significant plasma homocysteine elevations (van der Put, N. M., et al., supra, 1998) associated with a 50-60% decrement in enzyme activity (Weisberg, I., et al., Mol. Genet. Metab. 64:169-72, 1998). In the absence of other coding mutations elsewhere in the MTHFR gene, or of evidence for a mutant splice variant, our patient's deficient enzyme activity may be attributed to compound heterozygosity for the novel 1755G→A mutation with the prevalent 677C→T polymorphism on the same paternal chromosome, and the 1298A→C mutation on the maternal chromosome. It has recently been shown that when mutations causing severe MTHFR deficiency are expressed in cis with the common 677C→T variant the resultant phenotype is markedly aggravated (Goyette, P., et al., supra, 1994).

Approximately 45 million anesthetics are performed annually in North America, with nitrous oxide a significant component in about half (Orkin, F. K. and Thomas, S. J., “Scope of Modern Anesthetic Practice,” In: Miller, R. D., ed. Anesthesia, Philadelphia: Churchill Livingstone, 2577-85, 2000). Because of growing use (Peretz, B., et al., Int. Dent. J. 48:17-23, 1998; Keating, H. J., 3^{rd} and Kundrat, M., J. Pain Symptom Manage. 11:126-30, 1996; Luhmann, J. D., et al., Ann. Emerg. Med. 37:20-7, 2001; Castera, L., et al., Am. J. Gastroenterol. 96:1553-7, 2001; Krauss, B., Ann. Emerg. Med. 37:61-2, 2001), patients with both mild and severe abnormalities of folate cycle enzymes are increasingly likely to encounter nitrous oxide.

On the strength of the present findings, nitrous oxide use in patients with polymorphisms causing reduced activity of folate, cobalamin, methionine and homocysteine pathway enzymes is contraindicated.

TABLE 3
Familial polymorphisms.

| | | | | CBS | | |
|---|---|---|---|---|---|---|
| | | | | 68 bp | | |
| | MTHFR | MTHFR | MTHFR | inser- | MTR | MTRR |
| | 677C→T | 1298A→C | 1755G→A | tion | 2756A→G | 66A→G |
| Proband | C/T | A/C | G/A | WT | A/A | A/G |
| Brother | C/T | A/C | G/A | WT | A/A | A/G |
| Mother | C/T | A/C | G/G | WT | A/G | A/A |
| Father | T/T | A/A | G/A | WT | A/A | A/G |
| Uncle | C/T | A/C | G/G | WT | A/A | A/G |
| Uncle | T/T | A/A | G/A | WT | A/A | A/G |
| Aunt | T/T | A/A | G/A | WT | A/A | A/G |
| Aunt | C/C | C/C | G/G | WT | A/A | A/G |

TABLE 4
Oligonucleotide primers used for amplification and
sequencing of MTHFR Exons from genomic DNA.

| | | | | Product | | Annealing |
|---|---|---|---|---|---|---|
| | Primer | | | size | [Mg] | Temperature |
| Exon | Name | Primer Use | Primer Sequence | (bp) | mM | ° C. |
| 1 | MTHFR1F#2 | PCR, sequence | 5′-gcc act cag gtg tct tga tgt gtc gg-3′ | 384 | 3.0 | 64 |
| | MTHFR1R | PCR, sequence | 5′-tga cag ttt gct ccc cag gca c-3′³¹ | | | |
| 2 | MTHFR2F | PCR | 5′-gga agg cag tga cgg atg gta t-3′³⁰ | 373 | 1.5 | 60 |
| | MTHFR2R | PCR | 5′-acc aag ttc agg cta cca agt gg-3′³⁰ | | | |
| | MTHFR2F#2 | Sequence | 5′-tat ttc tcc tgg aac ctc tct tca-3′ | | | |
| | MTHFR2R#3 | Sequence | 5′-gcc tcc ggg aaa gcc aga acc-3′ | | | |
| 3 | MTHFR3F | PCR, sequence | 5′-ggg tga gac cca gtg act atg acc-3′ | 193 | 1.5 | 67.5 |
| | MTHFR3R | PCR, sequence | 5′-ccc tag ctc cat ccc cgc cac cag g-3′ | | | |
| 4 | MTHFR4F | PCR, sequence | 5′-ggt gga ggc cag cct ctc ctg-3′ | 285 | 1.5 | 67.5 |
| | MTHFR4R | PCR, sequence | 5′-gcg gtg aga gtg ggg tgg agg g-3′ | | | |
| 5 | MTHFR5F#2 | PCR, sequence | 5′-gct ggc cag cag ccg cca cag cc-3′ | 315 | 1.5 | 67.5 |
| | MTHFR5R#2 | PCR, sequence | 5′-gga tct ctg ggc cac tgc cct c-3′ | | | |
| 6 | MTHFR6F | PCR, sequence | 5′-tgc ttc cgg ctc cct cta gcc-3′³¹ | 250 | 1.5 | 60 |
| | MTHFR6R | PCR, sequence | 5′-cct ccc gct ccc aag aac aaa g-3′³¹ | | | |
| 7 | MTHFR7F | PCR, sequence | 5′-gcc ctc tgt cag gag tgt gcc c-3′ | 271 | 1.5 | 67.5 |
| | MTHFR7R | PCR | 5′-ggg cag ggg atg aac cag ggt ccc c-3′ | | | |
| | MTHFR7R#2 | Sequence | 5′-ggt ccc cac ttc cag cat cac-3′ | | | |
| 8 | MTHFR8F#2 | PCR, sequence | 5′-cag ggt gcc aaa cct gat ggt cgc c-3′ | 283 | 1.5 | 67.5 |
| | MTHFR8R#2 | PCR, sequence | 5′-cca cgg gtg ccg gtc aag aga gg-3′ | | | |
| 9 | MTHFR9F#2 | PCR, sequence | 5′-gtt ggt gac agg cac ctg tct ct-3′ | 182 | 1.5 | 67.5 |
| | MTHFR9R#2 | PCR, sequence | 5′-tgt tca acg aag ggc ctg gta c-3′ | | | |
| 10 | MTHFR10F | PCR, sequence | 5′-ggc cca ggt ctt acc ccc acc cc-3′ | 189 | 1.5 | 67.5 |
| | MTHFR10R | PCR, sequence | 5′-ggt ggg cgg ggc aag ctt gcc ccc-3′ | | | |
| 11 | MTHFR11F | PCR, sequence | 5′-gca tgt gtg cgt gtg tgc ggg gg-3′ | 516 | 1.5 | 67.5 |
| | MTHFR11R | PCR, sequence | 5′-cct ctg cag gag caa gtg ctc ccc-3′ | | | |

Primers used to amplify cDNA as seven overlapping fragments.

| | | | Product | | Annealing |
|---|---|---|---|---|---|
| | Primer | | size | [Mg] | Temperature |
| Fragment | Name | Primer Sequence | (bp) | mM | ° C. |
| 1 | X13F | 5′-cgg aca gcc ata gct gag gag c-3′^{a} | 1584 | 1.5 | 66 |
| | X14R | 5′-ggc tgg tct cag ccg cca gg-3′^{b} | | | |
| 2 | MTHFR 1F#2 | 5′- gcc act cag gtg tct tga tgt gtc gg-3′^{c} | 2206 | 1.5 | 64 |
| | MTHFR endR | 5′-cac tcc agt cta gct gcc att gtc-3′^{c} | | | |
| 3 | X17F | 5′-gcg aga gaa acg gag gct cc-3′^{a} | 977 | 1.5 | 67.5 |
| | X2R | 5′-cat ctg cac ctg cca gtc act gcc-3′^{a} | | | |
| 4 | X3F | 5′-cct ggc tgt gga ggc ctg atg ctg-3′^{a} | 1275 | 1.5 | 68.5 |
| | X4R | 5′-gga tcc ttg cga ctg cga gtg gct c-3′^{a} | | | |
| 5 | X5F | 5′-ggc cac aaa tca aag caa gg-3′^{a} | 1256 | 1.5 | 68.5 |
| | X6R | 5′-ctc ttt ggg tgg cag gca gcc g-3′^{a} | | | |
| 6 | X7F | 5′-cca gct act ctg tcc agg cca ctg-3′^{b} | 1274 | 1.5 | 68.5 |
| | X8R | 5′-ggc tca agc gat cta cct gcc ttg-3′^{b} | | | |
| 7 | X11F | 5′-ctc cat cag ctt atg gga tcc ttg tc-3′^{a} | 1174 | 1.5 | 67.5 |
| | X12R | 5′-ggc tga agc aga gga gtg atc tca gc-3′^{a} | | | |

Primers used to sequence the cDNA transcript

| | Primer | |
|---|---|---|
| Fragment | Name | Primer Sequence |
| Sense: | MTHFR 1F#2 | 5′-gcc act cag gtg tct tga tgt gtc gg-3′^{a} |
| | MTHFR 518F | 5′-gct gcc gtc agc gcc tgg agg ag-3′^{b} |
| | MTHFR 972F | 5′-gga cgt gat tga gcc aat caa aga c-3′^{c} |
| | MTHFR 1206F | 5′-gga aga tgt acg tcc cat ctt ctg g-3′^{c} |
| | MTHFR 1683F | 5′-gcg gaa gca ctt ctg caa gtg ctg-3′^{a} |
| Anti-sense: | MTHFR 515R | 5′-gtc atg tgc agg atg gtc tcc ag-3′^{a} |
| | MTHFR 1022R | 5′-cca tag ttg cgg atg gca gca tcg-3′^{a} |
| | MTHFR 1535R | 5′-tcc ttc agc agg ctg gtc tca gcc g-3′^{a} |
| | MTHFR 1806R | 5′-gac agc att cgg ctg cag ttc agg-3′^{a} |
| | MTHFR endR | 5′-cac tcc agt cta gct gcc att gtc-3′^{a} |

We claim:

1. A method for detection of susceptibility to nitrous oxide neurologic syndrome in a subject, comprising:
a) providing a sample from a subject, wherein said subject is a candidate for nitrous oxide exposure; and
b) detecting the presence or absence of folate, cobalamin, methionine and homocysteine pathway genetic polymorphisms in said sample, wherein the presence of a polymorphism indicates that the subject is susceptible to nitrous oxide neurologic syndrome.

2. The method of claim 1, wherein the sample is selected from the group consisting of a blood sample, a tissue sample, a urine sample, a cerebrospinal fluid sample, and an amniotic fluid sample.

3. The method of claim 1, wherein said subject is selected from the group consisting of an embryo, a fetus, a newborn animal, a young animal, and a mature animal.

4. The method of claim 1, wherein the subject is human.

5. The method of claim 1, wherein the detecting of step (b) is genomic testing.

6. The method of claim 5, wherein said genomic testing is testing for MTHFR polymorphisms.

7. The method of claim 6, wherein said MTHFR polymorphism is 1755G→A.

8. The method of claim 6, wherein said MTHFR polymorphisms are selected from a group consisting of 677C→T and 1298A→C.

9. The method of claim 5, wherein said genomic testing is testing for polymorphisms in the methionine synthase, methionine synthase reductase, and cystathionine β-synthase genes.

10. The method of claim 1, wherein said detecting is based on observations of peptides or proteins in the pathway.

11. The method of claim 10, wherein said detecting is an enzyme activity assay.

12. The method of claim 11, wherein said enzyme activity assay is MTHFR activity.

13. The method of claim 1, wherein said detecting is via the assay of a metabolite of the pathway.

14. The method of claim 13, wherein said metabolite is homocysteine.

15. The method of claim 13, wherein said metabolite is methionine.

16. The method of claim 13, wherein said metabolite is homocystine.

17. The method of claim 13, wherein said metabolite is cobalamin.

18. The method of claim 13, wherein said metabolite is folate.

19. A kit comprising a reagent for detecting the presence or absence of folate, cobalamin, methionine and homocysteine pathway genetic polymorphisms in a sample, wherein the reagent is a nucleic acid molecule comprising at least 11 nucleotides of the MTHFR, MTR, MTRR or CBS genes or their complement.

20. The kit of claim 19, further comprising instructions for using said kit for detecting the presence or absence of folate, cobalamin, methionine and homocysteine pathway genetic polymorphisms in a sample.

21. The kit of claim 19, wherein said instructions comprise instructions required by the U.S. Food and Drug Agency for in vitro diagnostic kits.

22. A method of diagnosing a mutation in the human 5,10-methylene tetrahydrofolate reductase gene comprising the step of examining a patient's 5,10-methylene tetrahydrofolate reductase gene and determining whether a polymorphism exists in residue 1755.

23. A method of diagnosing 5,10-methylene tetrahydrofolate reductase deficiency in a human patient comprising examining a patient's 5,10-methylene tetrahydrofolate reductase gene and determining whether a polymorphism exists.

24. The method of claim 22 where the polymorphism is 1775G→A.

25. The method of claim 22 comprising the additional step of examining the patient's 5,10-methylene tetrahydrofolate reductase gene for additional polymorphisms.

26. The method of claim 25 where the mutations are selected for the group consisting of 677C→T and 1298A→C.

27. The method of claim 25 wherein the mutations consist of a mutation selected from the group consisting of 677C→T and 1298A→C.

28. The method of claim 22 wherein the examination comprises amplifying the patient's 5,10-methylene tetrahydrofolate reductase gene.

29. The method of claim 22 wherein the examination comprises using a probe specific for the 1755G→A, mutation.

30. A gene probe useful to detect a mutation in the 5,10-methylene tetrahydrofolate reductase gene, comprising at least 11 nucleotides of SEQ ID NO:1 or the complement of this sequence, wherein the sequence includes residue 1755.

31. The probe of claim 30 additionally comprising at least 10 nucleotides selected from SEQ ID NO:2 and SEQ ID NO:3, wherein the sequence of the probe is such that the SEQ ID NO:2 or SEQ ID NO:3 sequences are chosen as naturally adjacent to the SEQ ID NO:1 sequence.


SEQUENCE LISTING (53 sequences)

SEQ ID NO: 1 - DNA, 118 bp, Homo sapiens
gtgaaaacatcaccaatgcccctgaactgcagccgaatgctgtcacttggggcatcttccctggcgagagatcatccagcccaccgtagtggatcccgtcagcttcatgttctggaag

SEQ ID NO: 2 - DNA, 56 bp, Homo sapiens
gacgaggcctttgccctgtggattgagcggtggggattcctggtcaacctggtgga

SEQ ID NO: 3 - DNA, 50 bp, Homo sapiens
gtaaaggagcgggggcaagcttgccccgcccacctggaaaaccgtgggga

SEQ ID NO: 4 - DNA, 11 bp, Artificial Sequence
  Synthetic
ttcatgttctg

SEQ ID NO: 5 - DNA, 24 bp, Artificial Sequence
  Synthetic
cccgtcagcttcatgttctggaag

SEQ ID NO: 6 - DNA, 11 bp, Artificial Sequence
  Synthetic
ttcatattctg

SEQ ID NO: 7 - DNA, 23 bp, Artificial Sequence
  Synthetic
ccgtcagcttcatattctggaac

SEQ ID NO: 8 - DNA, 26 bp, Artificial Sequence
  Synthetic
gccactcaggtgtcttgatgtgtcgg

SEQ ID NO: 9 - DNA, 22 bp, Artificial Sequence
  Synthetic
tgacagtttgctccccaggcac

SEQ ID NO: 10 - DNA, 22 bp, Artificial Sequence
  Synthetic
ggaaggcagtgacggatggtat

SEQ ID NO: 11 - DNA, 23 bp, Artificial Sequence
  Synthetic
accaagttcaggctaccaagtgg

SEQ ID NO: 12 - DNA, 24 bp, Artificial Sequence
  Synthetic
tatttctcctggaacctctcttca

SEQ ID NO: 13 - DNA, 21 bp, Artificial Sequence
  Synthetic
gcctccgggaaagccagaacc

SEQ ID NO: 14 - DNA, 24 bp, Artificial Sequence
  Synthetic
gggtgagacccagtgactatgacc

SEQ ID NO: 15 - DNA, 25 bp, Artificial Sequence
  Synthetic
ccctagctccatccccgccaccagg

SEQ ID NO: 16 - DNA, 21 bp, Artificial Sequence
  Synthetic
ggtggaggccagcctctcctg

SEQ ID NO: 17 - DNA, 22 bp, Artificial Sequence
  Synthetic
gcggtgagagtggggtggaggg

SEQ ID NO: 18 - DNA, 23 bp, Artificial Sequence
  Synthetic
gctggccagcagccgccacagcc

SEQ ID NO: 19 - DNA, 22 bp, Artificial Sequence
  Synthetic
ggatctctgggccactgccctc

SEQ ID NO: 20 - DNA, 21 bp, Artificial Sequence
  Synthetic
tgcttccggctccctctagcc

SEQ ID NO: 21 - DNA, 22 bp, Artificial Sequence
  Synthetic
cctcccgctcccaagaacaaag

SEQ ID NO: 22 - DNA, 22 bp, Artificial Sequence
  Synthetic
gccctctgtcaggagtgtgccc

SEQ ID NO: 23 - DNA, 25 bp, Artificial Sequence
  Synthetic
gggcaggggatgaaccagggtcccc

SEQ ID NO: 24 - DNA, 21 bp, Artificial Sequence
  Synthetic
ggtccccacttccagcatcac

SEQ ID NO: 25 - DNA, 25 bp, Artificial Sequence
  Synthetic
cagggtgccaaacctgatggtcgcc

SEQ ID NO: 26 - DNA, 23 bp, Artificial Sequence
  Synthetic
ccacgggtgccggtcaagagagg

SEQ ID NO: 27 - DNA, 23 bp, Artificial Sequence
  Synthetic
gttggtgacaggcacctgtctct

SEQ ID NO: 28 - DNA, 22 bp, Artificial Sequence
  Synthetic
tgttcaacgaagggcctggtac

SEQ ID NO: 29 - DNA, 23 bp, Artificial Sequence
  Synthetic
ggcccaggtcttacccccacccc

SEQ ID NO: 30 - DNA, 24 bp, Artificial Sequence
  Synthetic
ggtgggcggggcaagcttgccccc

SEQ ID NO: 31 - DNA, 23 bp, Artificial Sequence
  Synthetic
gcatgtgtgcgtgtgtgcggggg

SEQ ID NO: 32 - DNA, 24 bp, Artificial Sequence
  Synthetic
cctctgcaggagcaagtgctcccc

SEQ ID NO: 33 - DNA, 22 bp, Artificial Sequence
  Synthetic
cggacagccatagctgaggagc

SEQ ID NO: 34 - DNA, 20 bp, Artificial Sequence
  Synthetic
ggctggtctcagccgccagg

SEQ ID NO: 35 - DNA, 24 bp, Artificial Sequence
  Synthetic
cactccagtctagctgccattgtc

SEQ ID NO: 36 - DNA, 20 bp, Artificial Sequence
  Synthetic
gcgagagaaacggaggctcc

SEQ ID NO: 37 - DNA, 24 bp, Artificial Sequence
  Synthetic
catctgcacctgccagtcactgcc

SEQ ID NO: 38 - DNA, 24 bp, Artificial Sequence
  Synthetic
cctggctgtggaggcctgatgctg

SEQ ID NO: 39 - DNA, 25 bp, Artificial Sequence
  Synthetic
ggatccttgcgactgcgagtggctc

SEQ ID NO: 40 - DNA, 20 bp, Artificial Sequence
  Synthetic
ggccacaaatcaaagcaagg

SEQ ID NO: 41 - DNA, 22 bp, Artificial Sequence
  Synthetic
ctctttgggtggcaggcagccg

SEQ ID NO: 42 - DNA, 24 bp, Artificial Sequence
  Synthetic
ccagctactctgtccaggccactg

SEQ ID NO: 43 - DNA, 24 bp, Artificial Sequence
  Synthetic
ggctcaagcgatctacctgccttg

SEQ ID NO: 44 - DNA, 26 bp, Artificial Sequence
  Synthetic
ctccatcagcttatgggatccttgtc

SEQ ID NO: 45 - DNA, 26 bp, Artificial Sequence
  Synthetic
ggctgaagcagaggagtgatctcagc

SEQ ID NO: 46 - DNA, 23 bp, Artificial Sequence
  Synthetic
gctgccgtcagcgcctggaggag

SEQ ID NO: 47 - DNA, 25 bp, Artificial Sequence
  Synthetic
ggacgtgattgagccaatcaaagac

SEQ ID NO: 48 - DNA, 25 bp, Artificial Sequence
  Synthetic
ggaagatgtacgtcccatcttctgg

SEQ ID NO: 49 - DNA, 24 bp, Artificial Sequence
  Synthetic
gcggaagcacttctgcaagtgctg

SEQ ID NO: 50 - DNA, 23 bp, Artificial Sequence
  Synthetic
gtcatgtgcaggatggtctccag

SEQ ID NO: 51 - DNA, 24 bp, Artificial Sequence
  Synthetic
ccatagttgcggatggcagcatcg

SEQ ID NO: 52 - DNA, 25 bp, Artificial Sequence
  Synthetic
tccttcagcaggctggtctcagccg

SEQ ID NO: 53 - DNA, 24 bp, Artificial Sequence
  Synthetic
gacagcattcggctgcagttcagg